(19) United States
(12) Patent Application Publication
(10) Pub. No.: US 2022/0067919 A1
(43) Pub. Date: Mar. 3, 2022

(54) SYSTEM AND METHOD FOR IDENTIFYING A TUMOR OR LESION IN A PROBABILTY MAP

(71) Applicant: GE Precision Healthcare LLC, Wauwatosa, WI (US)

(72) Inventors: Krishna Seetharam Shriram, Bangalore (IN); Arathi Sreekumari, Bangalore (IN); Rakesh Mullick, Bangalore (IN)

(21) Appl. No.: 17/003,467

(22) Filed: Aug. 26, 2020

(51) Int. Cl. G06T 7/00; G06K 9/32; G06T 11/00

(52) U.S. Cl. CPC G06T 7/0012; G06K 9/3233; G06T 11/003; G06T 2207/20081; G06T 2207/30068; G06T 2207/30096; G06T 2207/10136

(57) ABSTRACT

The present disclosure relates to a system and method for identifying a tumor or lesion in a probability map. In accordance with certain embodiments, a method includes identifying, with a processor, a first region of interest in a first projection image, generating, with the processor, a first probability map from the first projection image and a second probability map from a second projection image, wherein the first probability map includes a second region of interest that has location that corresponds to a location of the first region of interest, interpolating the first probability map and the second probability map, thereby generating a probability volume, wherein the probability volume includes the second region of interest, and outputting, with the processor, a representation of the probability volume to a display.

### TECHNICAL FIELD

This disclosure relates to a system and method for identifying a tumor or lesion within a probability map and more particularly, to a system and method for identifying a tumor or lesion within a probability map generated from a plurality of projection images.

### BACKGROUND

Medical imaging devices (i.e., ultrasound, positron emission tomography (PET) scanner, computed tomography (CT) scanner, magnetic resonance imaging (MM) scanner, and X-ray machines, etc.) produce medial images (i.e., native Digital Imaging and Communications in Medicine (DICOM) images) representative of different parts of the body to identify tumors/lesions within the body.

The image data may be rendered into a 3D volume. Some approaches for identifying a tumor/lesion within the 3D volume require a clinician analyzing individual 2D slices that form the 3D volume to determine the presence of a tumor/lesion. Unfortunately, this process is time consuming as it requires the clinician to analyze several 2D slices. Another approach includes applying computer assistance detection (CAD) to the 3D volume. This approach applies deep learning techniques to the 3D volume to automatically identify regions of interest within the 3D volume that are indicative of a tumor/lesion. Unfortunately, such techniques require large amounts of processing power, consume large amounts of memory resources, and are time consuming as a computer system must analyze a large amount of data. Yet another approach includes applying CAD that includes deep learning techniques to individual 2D slices that form the 3D volume. While these approaches may be faster than the above 3D approaches, they may miss patterns indicative of a tumor/lesion as these patterns may not occur within an individual slice.

### SUMMARY

In one embodiment, the present disclosure provides a method. The method comprises identifying, with a processor, a first region of interest in a first projection image, generating, with the processor, a first probability map from the first projection image and a second probability map from a second projection image, wherein the first probability map includes a second region of interest that has location that corresponds to a location of the first region of interest, interpolating the first probability map and the second probability map, thereby generating a probability volume, wherein the probability volume includes the second region of interest, and outputting, with the processor, a representation of the probability volume to a display.

In another embodiment, the present disclosure provides a system. The system comprises a medical imaging system, a processor, and a computer readable storage medium. The computer readable storage medium is in communication with the processor. The computer readable storage medium stores program instructions. When the processor executes the program instructions cause the processor to: receive image data from the imaging system, generate a first and second set of two-dimensional images from the image data, generate a first projection image from the first set of two-dimensional images and a second projection image from the second set of two-dimensional images, identify a first region of interest in the first projection image, generate a first probability map from the first projection image and a second probability map from a second projection image, wherein the first probability map includes a second region of interest that has location that corresponds to a location of the first region of interest, interpolate the first probability map and the second probability map, thereby generating a probability volume, wherein the probability volume includes the second region of interest, and output a representation of the probability volume to a display.

In yet another embodiment, the present disclosure provides a computer readable storage medium. The computer readable storage medium comprises computer readable program instructions. The computer readable program instructions, when executed by a processor, cause the processor to: generate a three-dimensional volume from ultrasound data, wherein the three-dimensional volume includes a plurality of two-dimensional images, separate the plurality of two-dimensional images into a first set and a second set of two-dimensional images, generate a first projection image from the first set of two-dimensional images and a second projection image from the second set of two-dimensional images, identify a first region of interest in the first projection image, generate a first probability map from the first projection image and a second probability map from the second projection image, wherein the first probability map includes a second region of interest with a location that corresponds to a location of the first region of interest, generate a probability volume from the first and second probability maps, and identify a region of interest in the probability volume as a tumor or lesion.

### BRIEF DESCRIPTION OF THE DRAWINGS

Various aspects of this disclosure may be better understood upon reading the following detailed description upon reference to the drawings in which:

FIG. 1 is a schematic diagram of a medical imaging system in accordance with an exemplary embodiment;

FIG. 2 depicts an automated breast ultrasound system in accordance with an exemplary embodiment;

FIG. 3 depicts a scanning assembly of an automated breast ultrasound system in accordance with an exemplary embodiment;

FIG. 4 is a schematic diagram of a system for controlling an automated breast ultrasound system in accordance with an exemplary embodiment;

FIG. 5 is a schematic diagram of a communication module of an automated breast ultrasound system in accordance with an exemplary embodiment;

FIG. 6 is a schematic diagram of a cloud computing environment in accordance with an exemplary embodiment;

FIG. 7 is a flow chart of a method for identifying a tumor or lesion in a probability volume in accordance with an exemplary embodiment;

FIG. 8 depicts a ground truth mask in accordance with an exemplary embodiment;

FIG. 9 is a schematic diagram for separating images of a 3D volume in accordance with an exemplary embodiment;

FIG. 10 is another schematic diagram for separating images of a 3D volume in accordance with an exemplary embodiment;

FIG. 11 is another schematic diagram for separating images of a 3D volume in accordance with an exemplary embodiment;

FIG. 12 is a schematic diagram for generating a minimum intensity projection image from a plurality of two-dimensional images in accordance with an exemplary embodiment; and

FIG. 13 depicts a schematic diagram for generating a probability map in accordance with an exemplary embodiment.

The drawings illustrate specific aspects of the described components, systems, and methods for identifying a tumor or lesion within a probability volume. Together with the following description, the drawings demonstrate and explain the principles of the structures, methods, and principles described herein. In the drawings, the thickness and size of components may be exaggerated or otherwise modified for clarity. Well-known structures, materials, or operations are not shown or described in detail to avoid obscuring aspects of the described components, systems, and methods.

### DETAILED DESCRIPTION

One or more specific embodiments of the present disclosure are described below in order to provide a thorough understanding. These described embodiments are only examples of systems and methods for identifying a tumor or lesion within a probability volume generated from a plurality projection images. The skilled artisan will understand that specific details described in the embodiments can be modified when being placed into practice without deviating from the spirit of the present disclosure

When introducing elements of various embodiments of the present disclosure, the articles “a,” “an,” and “the” are intended to mean that there are one or more of the elements. The terms “first,” “second,” and the like, do not denote any order, quantity, or importance, but rather are used to distinguish one element from another. The terms “comprising,” “including,” and “having” are intended to be inclusive and mean that there may be additional elements other than the listed elements. As the terms “connected to,” “coupled to,” etc. are used herein, one object (i.e., a material, element, structure, number, etc.) can be connected to or coupled to another object regardless of whether the one object is directly connected or coupled to the other object or whether there are one or more intervening objects between the one object and the other object. In addition, it should be understood that references to “one embodiment” or “an embodiment” of the present disclosure are not intended to be interpreted as excluding the existence of additional embodiments that also incorporate the recited features.

Some embodiments of the present disclosure provide a system/method that generates a plurality of projection images from individual slices of a 3D volume and identify a tumor/lesion in a probability map and/or a probability volume generated from the plurality of projection images. Projection images may include minimum intensity projection images, maximum intensity projection images, average intensity projection images, median intensity projection image, etc. and may be obtained by projecting through multiple slices of the 3D volume. A system/method that identifies a tumor/lesion within a probability map and/or a probability volume may require less processing power than a system that analyzes a 3D volume as the probability map/volume includes less data than a 3D volume. Furthermore, a system/method that identifies a tumor/lesion within a probability map and/or a probability volume may be more accurate in identifying a tumor/lesion than a similar system that analyzes individual 2D slices as the probability map/volume contains data from several slices rather than one.

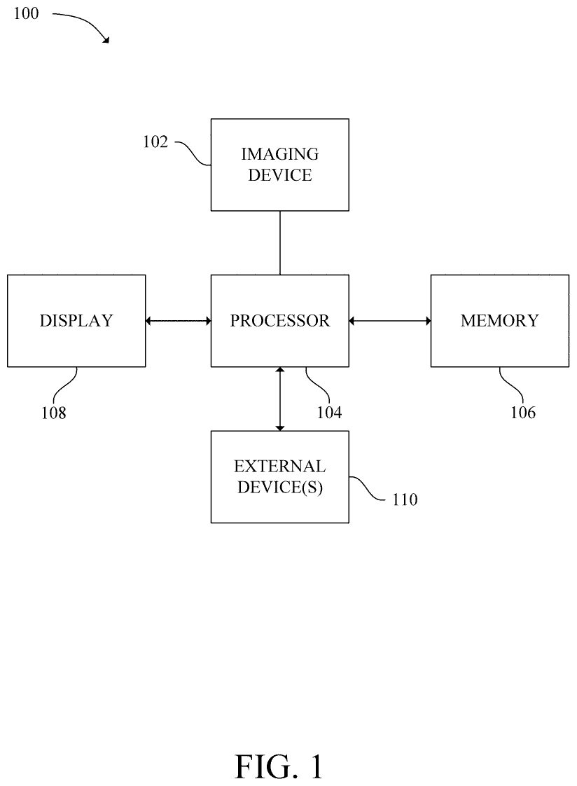

Referring now to FIG. 1, a medical imaging system 100 is shown in accordance with an exemplary embodiment. As illustrated in FIG. 1, in some embodiments, the medical imaging system 100 includes a medical imaging device 102, a processor 104, a system memory 106, a display 108, and one or more external devices 110.

The medical imaging device 102 may be any imaging device capable of capturing image data (i.e., PET, CT, Mill, X-ray machine, etc.) and capable of processing the captured image data into a 3D image volume. Particularly, the medical imaging device 102 may be an ultrasound device. The medical imaging device 102 is in communication with the processor 104 via a wired and/or a wireless connection thereby allowing the medical imaging device 102 to receive data from/send data to the processor 104. In one embodiment, the medical imaging device 102 may be connected to a network (i.e., a wide area network (WAN), a local area network (LAN), a public network (the Internet), etc.) which allows the medical imaging device 102 to transmit data to and/or receive data from the processor 104 when the processor 104 is connected to the same network. In another embodiment, the medical imaging device 102 is directly connected the processor 104 thereby allowing the medical imaging device 102 to transmit data directly to and receive data directly from the processor 104.

The processor 104 may be a processor of a computer system. A computer system may be any device/system that is capable of processing and transmitting data (i.e., tablet, handheld computing device, smart phone, personal computer, laptop, network computer, etc.). The processor 104 is in communication with the system memory 106. In one embodiment, the processor 104 may include a central processing unit (CPU). In another embodiment, the processor 104 may include other electronic components capable of executing computer readable program instructions, such as a digital signal processor, a field-programmable gate array (FPGA), or a graphics board. In yet another embodiment, the processor 104 may be configured as a graphical processing unit with parallel processing capabilities. In yet another embodiment, the processor 104 may include multiple electronic components capable of carrying out computer readable instructions. For example, the processor 104 may include two or more electronic components selected from a list of electronic components including: a CPU, a digital signal processor, an FPGA, and a graphics board.

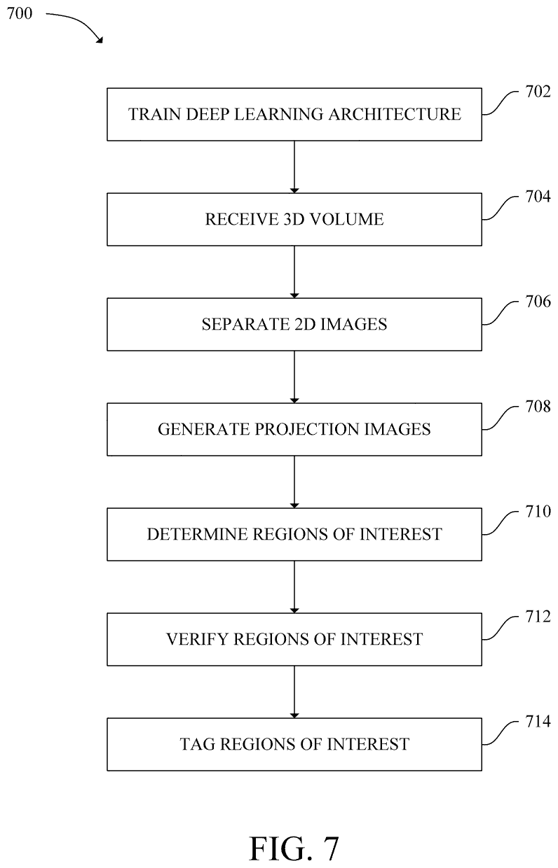

The system memory 106 is a computer readable storage medium. As used herein a computer readable storage medium is any device that stores computer readable program instructions for execution by a processor and is not construed as being transitory per se. Computer readable program instructions include programs, logic, data structures, modules, architecture etc. that when executed by a processor create a means for implementing functions/acts specified in FIG. 7. Computer readable program instructions when stored in a computer readable storage medium and executed by a processor direct a computer system and/or another device to function in a particular manner such that a computer readable storage medium comprises an article of manufacture. System memory as used herein includes volatile memory (i.e., random access memory (RAM) and dynamic RAM (DRAM)) and nonvolatile memory (i.e., flash memory, read-only memory (ROM), magnetic computer storage devices, etc.). In some embodiments, the system memory may further include cache.

The display 108 and the one or more external devices 110 are connected to and in communication with the processor 104 via an input/output (I/O) interface. The one or more external devices 110 include devices that allow a user to interact with/operate the medical imaging device 102 and/or a computer system with the processor 104. As used herein, external devices include, but are not limited to, a mouse, keyboard, and a touch screen.

The display 108 displays a graphical user interface (GUI). As used herein, a GUI includes editable data (i.e., patient data) and/or selectable icons. A user may use an external device to select an icon and/or edit the data. Selecting an icon causes a processor to execute computer readable program instructions stored in a computer readable storage medium which cause a processor to perform various tasks. For example, a user may use an external device 110 to select an icon which causes the processor 104 to control the medical imaging device 102 to capture DICOM images of a patient.

When the processor 104 executes computer readable program instructions to begin image acquisition, the processor 104 sends a signal to begin imaging to the imaging device 102. As the imaging device 102 moves, the imaging device 102 captures a plurality of 2D images (or “slices”) of an anatomical structure according to a number of techniques. The processor 104 may further execute computer readable program instructions to generate a 3D volume from the 2D slices according to a number of different techniques.

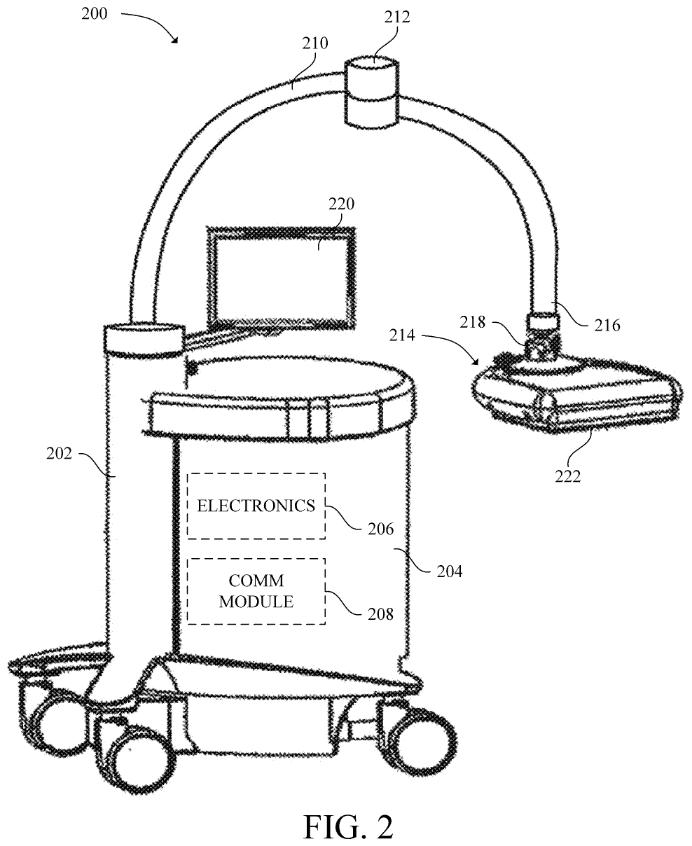

Referring now to FIG. 2 an automated breast ultrasound system (ABUS) system 200 is shown in accordance with an exemplary embodiment. The ABUS 200 may serve as the medical imaging system 100.

The ABUS 200 is a full-field breast ultrasound (FFBU) scanning apparatus. An FFBU may be used to image breast tissue in one or more planes. As will be discussed in further detail herein, a compression/scanning assembly of the ABUS 200 may include an at least partially conformable, substantially taut membrane or film sheet, an ultrasound transducer, and a transducer translation mechanism. One side of the taut membrane or film sheet compresses the breast. The transducer translation mechanism maintains the ultrasound transducer in contact with the other side of the film sheet while translating the ultrasound transducer thereacross to scan the breast. Prior to initiating the scanning, a user of the ABUS 200 may place an ultrasound transducer on a patient tissue and apply a downward force on the transducer to compress the tissue in order to properly image the tissue. The terms “scan” or “scanning” may be used herein to refer to acquiring data through the process of transmitting and receiving ultrasonic signals. The ABUS 200 compresses a breast in a generally chestward or head-on direction and ultrasonically scans the breast. In another example, the ABUS 200 may compress a breast along planes such as the craniocaudal (CC) plane, the mediolateral oblique (MLO) plane, or the like.

Although several examples herein are presented in the particular context of human breast ultrasound, it is to be appreciated that the present teachings are broadly applicable for facilitating ultrasound scanning of any externally accessible human or animal body part (i.e., abdomen, legs, feet, arms, neck, etc.). Moreover, although several examples herein are presented in the particular context of mechanized scanning (i.e., in which the ultrasound transducer is moved by a robot arm or other automated or semi-automated mechanism), it is to be appreciated that one or more aspects of the present teachings can be advantageously applied in a handheld scanning context.

FIG. 2 illustrates a perspective view of the ABUS 200. The ABUS 200 includes a frame 202, a housing 204 that contains electronics 206 and a communication module 208, a movable and adjustable support arm 210 (i.e., adjustable arm) including a hinge joint 212, a compression/scanning assembly 214 connected to a first end 216 of the adjustable arm 210 via a ball-and-socket connector (i.e., ball joint) 218, and a display 220 connected to the frame 202. The display 220 is coupled to the frame 202 at an interface where the adjustable arm 210 enters into the frame 202. As a result of being directly coupled to the frame 202 and not to the adjustable arm 210, the display 220 does not affect a weight of the adjustable arm 210 and a counterbalance mechanism of the adjustable arm 210. In one example, the display 220 is rotatable in a horizontal and lateral direction (i.e., rotatable around a central axis of the frame 202), but not vertically movable. In an alternate example, the display 220 may also be vertically movable. While FIG. 2 depicts the display 220 coupled to the frame 202, in other examples the display 220 may be coupled to a different component of the ABUS 200, such as coupled to the housing 204, or located remotely from the ABUS 200.

In one embodiment, the adjustable arm 210 is configured and adapted such that the compression/scanning assembly 214 is either (i) neutrally buoyant in space, or (ii) has a light net downward weight (i.e., 1-2 kg) for breast compression, while allowing for easy user manipulation. In alternate embodiments, the adjustable arm 210 is configured such that the compression/scanning assembly 214 is neutrally buoyant in space during positioning the scanner on the patient's tissue. Then, after positioning the compression/scanning assembly 214, internal components of the ABUS 200 may be adjusted to apply a desired downward weight for breast compression and increased image quality. In one example, the downward weight (i.e., force) may be in a range of 2-11 kg.

The adjustable arm 210 includes a hinge joint 212. The hinge joint 212 bisects the adjustable arm 210 into a first arm portion and a second arm portion. The first arm portion is coupled to the compression/scanning assembly 214 and the second arm portion is coupled to the frame 202. The hinge joint 212 allows the second arm portion to rotate relative to the second arm portion and the frame 202. For example, the hinge joint 212 allows the compression/scanning assembly 214 to translate laterally and horizontally, but not vertically, with respect to the second arm portion and the frame 202. In this way, the compression/scanning assembly 214 may rotate toward or away from the frame 202. However, the hinge joint 212 is configured to allow the entire adjustable arm 210 (i.e., the first arm portion and the second arm portion) to move vertically together as one piece (i.e., translate upwards and downwards with the frame 202).

The compression/scanning assembly 214 comprises an at least partially conformable membrane 222 in a substantially taut state for compressing a breast, the membrane 222 having a bottom surface contacting the breast while a transducer is swept across a top surface thereof to scan the breast. In one example, the membrane 222 is a taut fabric sheet.

Optionally, the adjustable arm 210 may comprise potentiometers (not shown) to allow position and orientation sensing for the compression/scanning assembly 214, or other types of position and orientation sensing (i.e., gyroscopic, magnetic, optical, radio frequency (RF)) can be used.

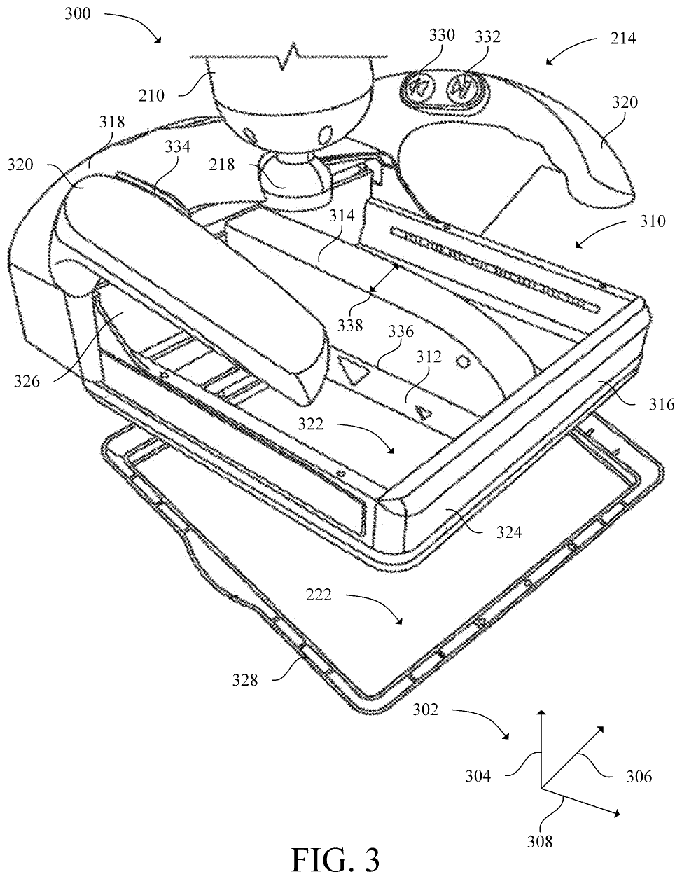

FIG. 3 shows a schematic 300 of an isometric view of the scanning assembly 214 coupled to the adjustable arm 210. The schematic 300 includes a coordinate system 302 including a vertical axis 304, horizontal axis 306, and a lateral axis 308.

The scanning assembly 214 includes a housing 310, a transducer module 312, and a module receiver 314. The housing 310 includes a frame 316 and a handle portion 318, the handle portion 318 including two handles 320. The two handles 320 are opposite one another across a lateral axis of the scanning assembly 214, the lateral axis is centered at the adjustable arm 210 and defined with respect to the lateral axis 308. The frame 316 is rectangular-shaped with an interior perimeter of the frame 316 defining an opening 322. The opening 322 provides a space (i.e., void volume) for translating the module receiver 314 and the transducer module 312 during a scanning procedure. In another example, the frame 316 may be another shape, such as square with a square-shaped opening 322. Additionally, the frame 316 has a thickness defined between the interior perimeter and an exterior perimeter of the frame 316.

The frame 316 includes four sets of side walls (i.e., the set including an interior side wall and an exterior side wall, the interior side walls defining the opening 322). Specifically, the frame 316 includes a front side wall 324 and a back side wall 326, the back side wall 326 directly coupled to the handle portion 318 of the housing 310 and the front side wall 324 opposite the back side wall 326 with respect to the horizontal axis 306. The frame 316 further includes a right side wall and a left side wall, the respective side walls opposite from one another and both in a plane defined by the vertical axis 304 and the lateral axis 308.

The frame 316 of the housing 310 further includes a top side and a bottom side, the top side and bottom side defined relative to the vertical axis 304. The top side faces the adjustable arm 210. A membrane 222 is disposed across the opening 322. More specifically, the membrane 222 is coupled to the bottom side of the frame 316. In one example, the membrane 222 is a membranous sheet maintained taut across the opening 322. The membrane 222 may be a flexible but non-stretchable material that is thin, water-resistant, durable, highly acoustically transparent, chemically resistant, and/or biocompatible. As discussed above, the bottom surface of the membrane 222 may contact a tissue (i.e., such as a breast) during scanning and a top surface of the membrane 222 may at least partially contact the transducer module 312 during scanning. As shown in FIG. 3, the membrane 222 is permanently coupled to a hard-shell clamping portion 328 around a perimeter of the membrane 222. The clamping portion 328 couples to the bottom side of the frame 316. In one example, the clamping portion 328 may snap to a lip on the bottom side of the frame 316 of the housing 310 such that the membrane 222 does not become uncoupled during scanning but is still removably coupled to the frame 316. In other embodiments, the membrane 222 may not be permanently coupled to a hard-shell clamping portion 328, and thus the membrane 222 may not couple to the frame 316 via the hard-shell clamping portion 328. Instead, the membrane 222 may be directly and removably coupled to the frame 316.

The handle portion 318 of the housing 310 includes two handles 320 for moving the scanning assembly 214 in space and positioning the scanning assembly 214 on a tissue (i.e., on a patient). In alternate embodiments, the housing 310 may not include handles 320. In one example, the handles 320 may be formed as one piece with the frame 316 of the housing 310. In another example, the handles 320 and the frame 316 may be formed separately and then mechanically coupled together to form the entire housing 310 of the scanning assembly 214.

As shown in FIG. 3, the scanning assembly 214 is coupled to the adjustable arm 210 through the ball joint 218 (i.e., ball-and-socket connector). The ball joint 218 is movable in multiple directions. For example, the ball joint 218 provides rotational movement of the scanning assembly 214 relative to the adjustable arm 210. The ball joint 218 includes a locking mechanism for locking the ball joint 218 in place and thereby maintaining the scanning assembly 214 stationary relative to the adjustable arm 210.

Additionally, as shown in FIG. 3, the handles 320 of the handle portion 318 include buttons for controlling scanning and adjusting the scanning assembly 214. Specifically, a first handle of the handles 320 includes a first weight adjustment button 330 and a second weight adjustment button 332. The first weight adjustment button 330 may decrease a load applied to the scanning assembly 214 from the adjustable arm 210. The second weight adjustment button 332 may increase the load applied to the scanning assembly 214 from the adjustable arm 210. Increasing the load applied to the scanning assembly 214 may increase an amount of pressure and compression applied to the tissue on which the scanning assembly 214 is placed. Further, increasing the load applied to the scanning assembly 214 increases the effective weight of the scanning assembly 214 on the tissue to be scanned. In one example, increasing the load may compress the tissue, such as a breast, of a patient. In this way, varying amounts of pressure (i.e., load) may be applied consistently with the scanning assembly 214 during scanning in order to obtain a quality image with the transducer module 312.

Before a scanning procedure, a user (i.e., ultrasound technician or physician) may position the scanning assembly 214 on a patient or tissue. Once the scanning assembly 214 is positioned correctly, the user may adjust the weight of the scanning assembly 214 on the patient (i.e., adjust the amount of compression) using the first weight adjustment button 330 and/or the second weight adjustment button 332. A user may then initiate a scanning procedure with additional controls on the handle portion 318 of the housing 310. For example, as shown in FIG. 3, a second handle of the handles 320 includes two additional buttons 334 (not individually shown). The two additional buttons 334 may include a first button to initiate scanning (i.e., once the scanning assembly 214 has been placed on the tissue/patient and the amount of compression has been selected) and a second button to stop scanning. In one example, upon selecting the first button, the ball joint 218 may lock, thereby stopping lateral and horizontal movement of the scanning assembly 214.

The module receiver 314 is positioned within the housing 310. Specifically, the module receiver 314 is mechanically coupled to a first end of the housing 310 at the back side wall 326 of the frame 316, the first end closer to the adjustable arm 210 than a second end of the housing 310. The second end of the housing 310 is at the front side wall 324 of the frame 316. The module receiver 314 is coupled to the transducer module 312. The module receiver 314 is coupled to the first end via a protrusion of the module receiver 314, the protrusion coupled to an actuator (not shown) of the module receiver 314.

The housing 310 is configured to remain stationary during scanning. In other words, upon adjusting a weight applied to the scanning assembly 214 through the adjustable arm 210 and then locking the ball joint 218, the housing 310 may remain in a stationary position without translating in the horizontal or lateral directions. However, the housing 310 may still translate vertically with vertical movement of the adjustable arm 210.

Conversely, the module receiver 314 is configured to translate with respect to the housing 310 during scanning. As shown in FIG. 3, the module receiver 314 translates horizontally, along the horizontal axis 306, with respect to the housing 310. The actuator of the module receiver 314 may slide the module receiver 314 along a top surface of the first end of the housing 310.

The transducer module 312 is removably coupled with the module receiver 314. As a result, during scanning, the transducer module 312 translates horizontally with the module receiver 314. During scanning, transducer module 312 sweeps horizontally across the breast under control of the module receiver 314 while a contact surface of the transducer module 312 is in contact with the membrane 222. The transducer module 312 and the module receiver 314 are coupled together at a module interface 336. The module receiver 314 has a width 338 which is the same as a width of the transducer module 312. In alternate embodiments, the width 338 of the module receiver 314 may not be the same as the width of the transducer module 312. In some embodiments, the module interface 336 includes a connection between the transducer module 312 and the module receiver 314, the connection including a mechanical and electrical connection.

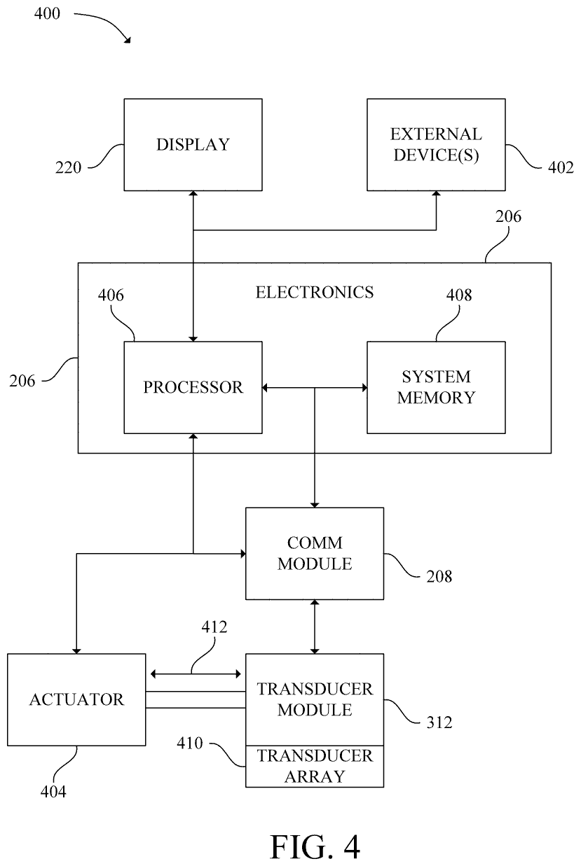

FIG. 4 is a schematic diagram of a system 400 for controlling the ABUS 200. The system 400 includes the electronics 206, the communication module 208, the display 220, the transducer module 312, one or more external device(s) 402, and an actuator 404.

In some embodiments, as depicted in FIG. 4, the electronics 206 include a processor 406 and a system memory 408. In other embodiments, the processor 406 and system memory 408 may be a processor and system memory of a computer system that is separate and remote from the ABUS 200. The processor 406 is in communication with the transducer module 312 via a wire or wireless connection thereby allowing the transducer module 312 to receive data from/send data to the processor 406. In one embodiment, the transducer module 312 may be connected to a network which allows the transducer module 312 to transmit data to and/or receive data from the processor 406 when the processor 406 is connected to the same network. In another embodiment, the transducer module 312 is directly connected the processor 406 thereby allowing the transducer module 312 to transmit data directly to and receive data directly from the processor 406.

The processor 406 is also in communication with the system memory 408. In one embodiment, the processor 406 may include a CPU. In another embodiment, the processor 406 may include other electronic components capable of executing computer readable program instructions. In yet another embodiment, the processor 406 may be configured as a graphical processing unit with parallel processing capabilities. In yet another embodiment, the processor 406 may include multiple electronic components capable of carrying out computer readable instructions. The system memory 408 is a computer readable storage medium.

The display 220 and the one or more external devices (i.e., keyboard, mouse, touch screen, etc.) 402 are connected to and in communication with the processor 406 via an input/output (I/O) interface. The one or more external devices 402 allow a user to interact with/operate the ABUS 200, the transducer module 312 and/or a computer system with the processor 406.

The transducer module 312 includes a transducer array 410. The transducer array 410 includes, in some embodiments, an array of elements that emit and capture ultrasonic signals. In one embodiment the elements may be arranged in a single dimension (a “one-dimensional-transducer array”). In another embodiment the elements may be arranged two dimensions (a “two-dimensional transducer array”). Furthermore, the transducer array 410 may be a linear array of one or several elements, a curved array, a phased array, a linear phased array, a curved phased array, etc. The transducer array 410 may be a 1D transducer array, a 1.25D transducer array, a 1.5D transducer array, a 1.75D transducer array, or a 2D array according to various embodiments. The transducer array 410 may be in a mechanical 3D or 4D probe that is configured to mechanically sweep or rotate the transducer array 410 with respect to the transducer module 312. Instead of an array of elements, other embodiments may have a single transducer element.

The transducer array 410 is in communication with the communication module 208. The communication module 208 connects the transducer module 312 to the processor 406 via a wired and/or a wireless connection. The processor 406 may execute computer readable program instructions stored in the system memory 408 which may cause the transducer array 410 to acquire ultrasound data, activate a subset of elements, and a emit an ultrasonic beam in a particular shape.

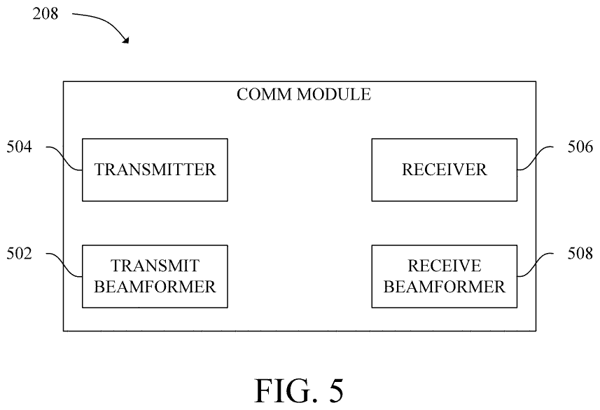

Referring now to FIG. 5, the communication module 208 is shown in accordance with an exemplary embodiment. As shown in FIG. 5, in some embodiments, the communication module 208 includes a transmit beamformer 502, a transmitter 504, a receiver 506, and a receive beamformer 508. With reference to FIGS. 4 and 5, when the processor 406 executes computer readable program instructions to begin image acquisition, the processor 406 sends a signal to begin image acquisition to the transmit beamformer 502. The transmit beamformer 502 processes the signal and sends a signal indicative of imaging parameters to the transmitter 504. In response, the transmitter 504 sends a signal to generate ultrasonic waves to the transducer array 410. Elements of the transducer array 410 then generate and output pulsed ultrasonic waves into the body of a patient. The pulsed ultrasonic waves reflect off features within the body (i.e., blood cells, muscular tissue, etc.) thereby producing echoes that return to and are captured by the elements. The elements convert the captured echoes into electrical signals which are sent to the receiver 506. In response, the receiver 506 sends the electrical signals to the receive beamformer 508 which processes the electrical signal into ultrasound image data. The receive beamformer 508 then sends the ultrasound image data to the processor 406. The transducer module 312 may contain all or part of the electronic circuitry to do all or part of the transmit and/or the receive beamforming. For example, all or part of the communication module 208 may be situated within the transducer module 312.

When the processor 406 executes computer readable program instructions to perform a scan, the instructions cause the processor 406 to send a signal to the actuator 404 to move the transducer module 312 in the direction 412. In response, the actuator 404 automatically moves the transducer module 312 while the with the transducer array 410 captures ultrasound data.

In one embodiment, the processor 406 may process the ultrasound data into a plurality of 2D slices wherein each image corresponds to a pulsed ultrasonic wave. In this embodiment, when the ultrasound probe 406 is moved during a scan, each slice may include a different segment of an anatomical structure. In some embodiments, the processor 406 outputs one or more slice to the display 220. In other embodiments, the processor 406 may further process the slices to generate a 3D volume and outputs the 3D volume to the display 220.

The processor 406 may further execute computer readable program instructions which cause the processor 406 to perform one or more processing operations on the ultrasound data according to a plurality of selectable ultrasound modalities. The ultrasound data may be processed in real-time during a scan as the echo signals are received. As used herein, the term “real-time” includes a procedure that is performed without any intentional delay. For example, the transducer module 312 may acquire ultrasound data at a real-time rate of 7-20 volumes/second. The transducer module 312 may acquire 2D data of one or more planes at a faster rate. It is understood that real-time volume-rate is dependent on the length of time it takes to acquire a volume of data. Accordingly, when acquiring a large volume of data, the real-time volume-rate may be slower.

The ultrasound data may be temporarily stored in a buffer (not shown) during a scan and processed in less than real-time in a live or off-line operation. In one embodiment, wherein the processor 406 includes a first processor 406 and a second processor 406, the first processor 406 may execute computer readable program instructions that cause the first processor 406 to demodulate radio frequency (RF) data and the second processor 406, simultaneously, may execute computer readable program instructions that cause the second processor 406 to further process the ultrasound data prior to displaying an image.

The transducer module 312 may continuously acquire data at, for example, a volume-rate of 10-30 hertz (Hz). Images generated from the ultrasound data may be refreshed at a similar fame-rate. Other embodiments may acquire and display data at different rates (i.e., greater than 30 Hz or less than 10 Hz) depending on the size of the volume and the intended application. In one embodiment, system memory 408 stores at least several seconds of volumes of ultrasound data. The volumes are stored in a manner to facilitate retrieval thereof according to order or time of acquisition.

In various embodiments, the processor 406 may execute various computer readable program instructions to process the ultrasound data by other different mode-related modules (i.e., B-mode, Color Doppler, M-Mode, Color M-mode, spectral Doppler, Elastography, TVI, strain, strain rate, etc.) to form 2D or 3D ultrasound data. For example, one or more modules may generate B-mode, color Doppler, M-mode, spectral Doppler, Elastography, TVI, strain rate, strain, etc. Image lines and/or volumes are stored in the system memory 408 with timing information indicating a time at which the data was acquired. The modules may include, for example, a scan conversion mode to perform scan conversion operations to convert the image volumes from beam space coordinates to display space coordinates. A video processor module may read the image volumes stored in the system memory 408 and cause the processor 406 to generate and output an image to the display 220 in real-time while a scan is being carried out.

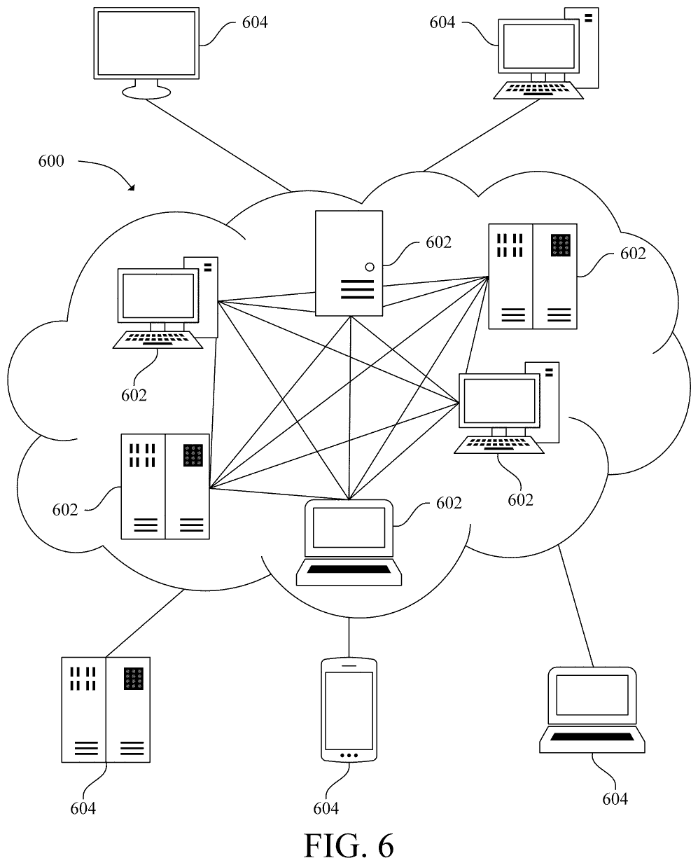

Referring now to FIG. 6, a cloud computing environment 600 is shown in accordance with an exemplary embodiment. As illustrated in FIG. 6, in some embodiments, the cloud computing environment 600 includes one or more nodes 602. Each node 602 may include a computer system/server (i.e., a personal computer system, a server computer system, a mainframe computer system, etc.). The nodes 602 may communicate with one another and may be grouped into one or more networks. Each node 602 may include a computer readable storage medium and a processor that executes instructions stored in the computer readable storage medium. As further illustrated in FIG. 6 one more devices (or systems) 604 may be connected to the cloud computing environment 600. The one or more devices 604 may be connected to a same or different network (i.e., LAN, WAN public network, etc.). The one or more devices 604 may include the medical imaging system 100 and the ABUS 200. One or more nodes 602 may communicate with the devices 604 thereby allowing the nodes 602 to provide software services to the devices 604.

In some embodiments, the processor 104 or the processor 406 may output a generated image to a computer readable storage medium of a picture archiving communications system (PACS). A PACS stores images generated by medical imaging devices and allows a user of a computer system to access the medical images. The computer readable storage medium may be one or more computer readable storage mediums and may be a computer readable storage medium of a node 602 and/or another device 604.

A processor of a node 602 or another device 604 may execute computer readable instructions in order to train a deep learning architecture. A deep learning architecture applies a set of algorithms to model high-level abstractions in data using multiple processing layers. Deep learning training includes training the deep learning architecture to identify features within in an image (i.e., a projection image) based on similar features in a plurality of training images. “Supervised learning” is a deep learning training method in which the training dataset includes only images with already classified data. That is, the training data set includes images wherein a clinician has previously identified structures of interest (i.e., tumors, lesions, etc.) within each training image. “Semi-supervised learning” is a deep learning training method in which the training dataset includes some images with already classified data and some images without classified data. “Unsupervised learning” is a deep learning training method in which the training data set includes only images without classified data but identifies abnormalities within the data set. “Transfer learning” is a deep learning training method in which information stored in a computer readable storage medium that was used to solve a first problem is used to solve a problem a second problem of a same or similar nature as the first problem.

Deep learning operates on the understanding that datasets include high level features which include low level features. While examining an image, for example, rather than looking for an object (i.e., a tumor, lesion, structure, etc.) within an image, a deep learning architecture looks for edges which form motifs which form parts, which form the object being sought based on learned observable features. Learned observable features include objects and quantifiable regularities learned by the deep learning architecture during supervised learning. A deep learning architecture provided with a large set of well classified data is better equipped to distinguish and extract the features pertinent to successful classification of new data.

A deep learning architecture that utilizes transfer learning may properly connect data features to certain classifications affirmed by a human expert. Conversely, the same deep learning architecture can, when informed of an incorrect classification by a human expert, update the parameters for classification. Settings and/or other configuration information, for example, can be guided by learned use of settings and/or other configuration information, and, as a system is used more (i.e., repeatedly and/or by multiple users), a number of variations and/or other possibilities for settings and/or other configuration information can be reduced for a given situation. Deep learning architecture can be trained on a set of expert classified data. This set of data builds the first parameters for the architecture and is the stage of supervised learning. During the stage of supervised learning, the architecture can be tested whether the desired behavior has been achieved.

Once a desired behavior has been achieved (i.e., the architecture has been trained to operate according to a specified threshold, etc.), the architecture can be deployed for use (i.e., testing the architecture with “real” data, etc.). During operation, architecture classifications can be confirmed or denied (i.e., by an expert user, expert system, reference database, etc.) to continue to improve architecture behavior. The architecture is then in a state of transfer learning, as parameters for classification that determine architecture behavior are updated based on ongoing interactions. In certain examples, the architecture can provide direct feedback to another process. In certain examples, the architecture outputs data that is buffered (i.e., via the cloud, etc.) and validated before it is provided to another process.

Deep learning architecture can be applied via a CAD to analyze medical images. The images may be stored in a PACS and/or generated by the medical imaging system 100 or the ABUS 200. Particularly, deep learning can be used to analyze projection images (i.e., minimum intensity projection image, maximum intensity projection image, average intensity projection image, median intensity projection image, etc.) generated from a 3D volume, probability maps generated from the projection images, and probability volumes generated from the probability maps.

Referring now to FIG. 7 a flow chart of a method 700 for identifying a region of interest within a probability map is shown in accordance with an exemplary embodiment. Various aspects of the method 700 may be carried out by a “configured processor.” As used herein a configured processor is a processor that is configured according to an aspect of the present disclosure. A configured processor(s) may be the processor 104, the processor 406, a processor of a node 602, or a processor of another device 604. A configured processor executes various computer readable instructions to perform the steps of the method 700. The computer readable instructions that, when executed by a configured processor, cause a configured processor to perform the steps of the method 700 may be stored in the system memory 106, the system memory 408, memory of a node 402, or memory of another device 404. The technical effect of the method 700 is to identify a region of interest as a tumor or lesion.

At 702, the configured processor trains a deep learning architecture with a plurality of 2D projection images (“the training dataset”). The projection images include, but are not limited to, minimum intensity projection images, maximum intensity projection images, average projection intensity images, and median intensity projection images. The deep learning architecture applies supervised, semi-supervised or unsupervised learning to determine one or more regions of interest within the training dataset. Furthermore, at 702, the configured processor compares the identified regions of interest to a ground truth mask. As used herein, a ground truth mask is an image or volume that includes accurately identified regions of interest. The regions of interest in the ground truth mask are regions of interest identified by a clinician. During training, the configured processor updates weights of the deep learning architecture as a function of the regions of interest identified in the ground truth mask.

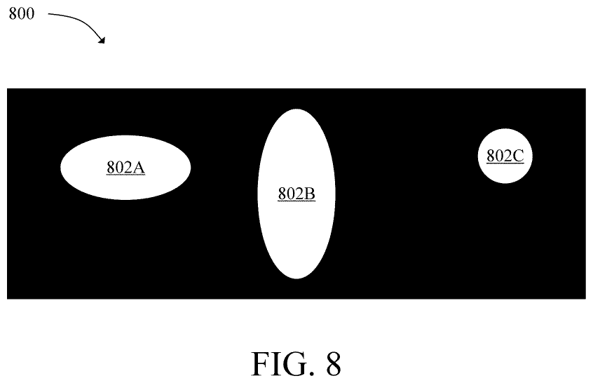

Briefly turning to FIG. 8, a ground truth mask 800 is shown in accordance with an exemplary embodiment. The ground truth mask 800 includes a first interest 802A, a second region of interest 802B, and a third region of interest 802C. While FIG. 8 depicts the ground truth mask 800 as including three regions of interest, it is understood that a ground truth mask may include more or less than three regions of interest. The regions of interest 802 correspond to regions that are classified as a tumor or lesion by a clinician within images of a training dataset.

Returning to FIG. 7, furthermore, at 702, the configured processor applies the deep learning architecture to a test data set and, with the deep learning architecture, identifies regions of interest within the test data set. The configured processor then checks the accuracy of the deep learning against a ground truth mask. If the deep learning architecture does not achieve a threshold level of accuracy (i.e., 80% accuracy, 90% accuracy, 95% accuracy, etc.), then the configured processor continues to train the deep learning architecture. When the deep learning architecture achieves the desired accuracy, the deep learning architecture is a trained deep learning architecture that can be applied to other data sets that do not include previously identified tumors or lesions.

At 704, the configured processor receives a 3D volume from the medical imaging system 100, the ABUS 200 or a PACS. A 3D volume comprises a plurality of 2D images. When the medical imaging system 100 or the ABUS 200 generates the 3D volume, each 2D image is a slice of an anatomical structure that is captured during an imaging procedure.

At 706, the configured processor separates the 2D images of the received 3D volume into a plurality of sets of 2D images. In some embodiments, each set may have a same number of 2D images. In other embodiments, each set may have a different number of 2D images.

Furthermore, in some embodiments, some sets may include a same 2D image. In other embodiments, each set may include different 2D images.

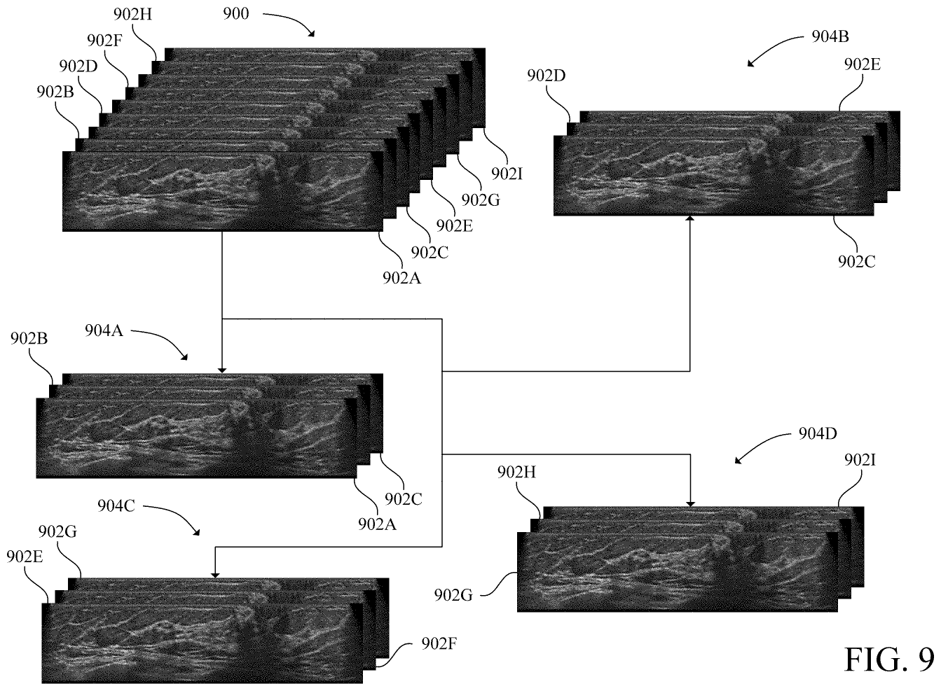

Briefly turning to FIG. 9, a 3D volume 900 is shown in accordance with an exemplary embodiment. As illustrated in FIG. 9, the 3D volume 900 includes a first 2D image 902A, a second 2D image 902B, a third 2D image 902C, . . . , and a ninth 2D image 902I. In this embodiment, the configured processor separates the 2D images 902 in the 3D volume 900 into a first set 904A, a second set 904B, a third set 904C, and a fourth set 904D of 2D images. The first set 904A includes the first 2D image 902A, the second 2D image 902B, and the third 2D image 902C. The second set 904B includes the third 2D image 902C, the fourth 2D image 902D, and the fifth 2D image 902E. The third set 904C includes the fifth 2D image 902E, the sixth 2D image 902F, and the seventh 2D image 902G. The fourth set 904D includes the seventh 2D image 902G, the eighth 2D image 902H, and the ninth 2D image 902I.

Each set 904 includes neighboring 2D images 902. That is, any given 2D image 902 in a given set 904 anatomically neighbors the 2D image 902 that immediately precedes and/or follows the given 2D image 902 in the given set 904. For example, the fourth image 902D neighbors the third 2D image 902C and the fifth 2D image 902E as the third 2D image 902C immediately precedes the fourth 2D image 902D and the fifth 2D image 902E immediately follows the fourth 2D image 902D. Furthermore, in this embodiment, each set 904 includes at least one 2D image 902 that appears in another set 904. For example, the first set 904A and the second set 904B include the third 2D image 902C.

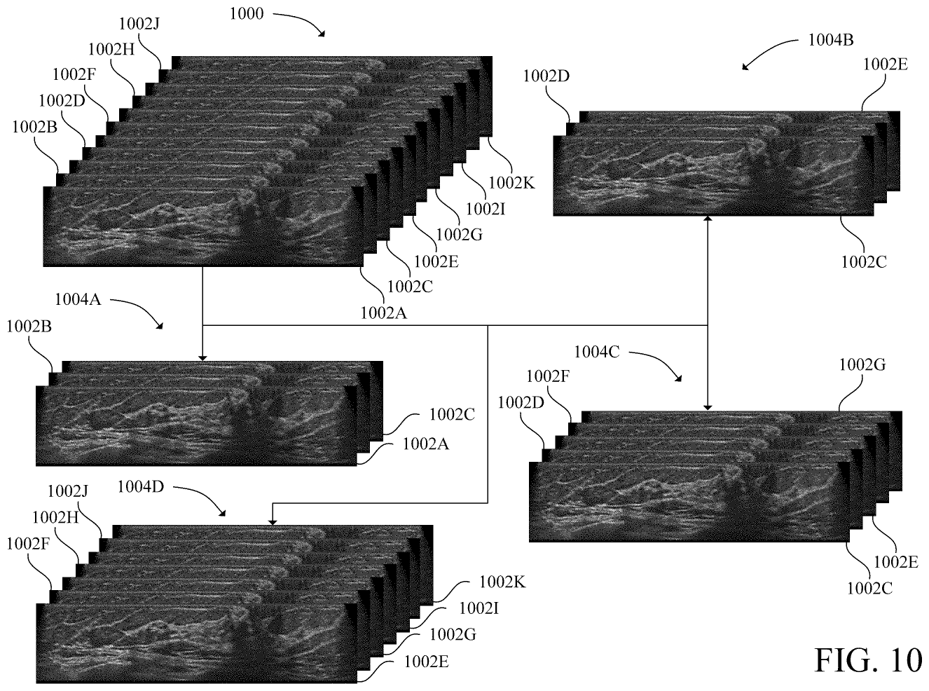

Referring now to FIG. 10, a 3D volume 1000 is shown in accordance with an exemplary embodiment. As illustrated in FIG. 10, the 3D volume 1000 includes a first 2D image 1002A, a second 2D image 1002B, a third 2D image 1002C, . . . , and an eleventh 2D image 1002K. In this embodiment, the configured processor separates the 3D volume 1000 into a first set 1004A, a second set 1004B, a third set 1004C, and a fourth set 1004D. The first set 1004A includes the first 2D image 1002A, the second 2D image 1002B, and the third 2D image 1002C. The second set 1004B includes the third 2D image 1002C, the fourth 2D 1002D, and the fifth 2D image 1002E. The third set 1004C includes the third 2D image 1002C, the fourth 2D image 1002D, the fifth 2D image 1002E, the sixth 2D image 1002F, and the seventh 2D image 10002G. The fourth set 1004D includes the third fifth 2D image 1002E, the sixth 2D image 1002F, the seventh 2D image 1002G, the eight 2D image 1002H, the ninth 2D image 1002I, the tenth 2D image 1002J, and the eleventh 2D image 1002K. As discussed above with reference to FIG. 9, each set 1004 includes neighboring 2D images 1002.

In this embodiment, each set 1004 may include a different number of 2D images 1002. For example, the first set 1004A includes three 2D images 1002 whereas the third set 1004C includes five 2D images 1002. Furthermore, each set 1004 may include more than one 2D image 1002 that appears in another set 1004. For example, the third set 1004C and the fourth set 1004D include the fifth 2D image 1002E, the sixth 2D image 1002F, and the seventh 2D image 1002G.

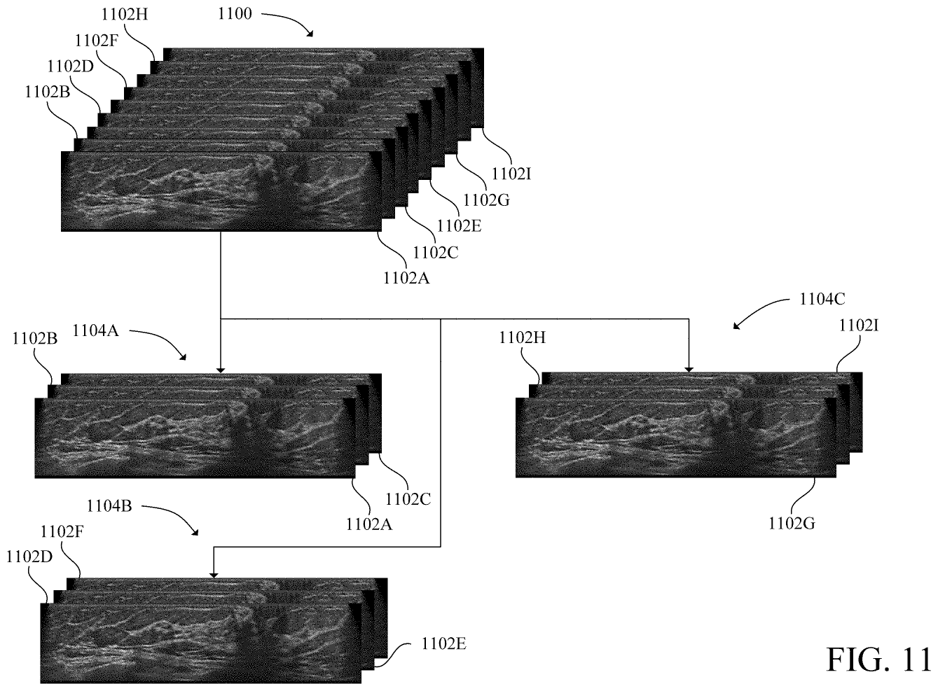

Referring now to FIG. 11, a 3D volume 1100 is shown in accordance with an exemplary embodiment. As illustrated in FIG. 11, the 3D volume 1100 includes a first 2D image 1102A, a second 2D image 1102B, a third 2D image 1102C, . . . , and a ninth 2D image 1102I. In this embodiment, the configured processor separates the 2D images 1102 in the 3D volume 1100 into a first set 1104A, a second set 1104B, and a third set 1104C. The first set 1104A includes the first 2D image 1102A, the second 2D image 1102B, and the third 2D image 1102C. The second set 1104B includes the fourth 2D image 1102D, the fifth 2D image 1102E, and the sixth 2D image 1102F. The third set 1104C includes the seventh 2D image 1102G, the eight 2D image 1102H, and the ninth 2D image 1102I. As discussed with reference to FIG. 9, each set 1104 includes neighboring 2D images 1102. Furthermore, in this embodiment, each set 1104 includes different 2D image 1102. That is, no two sets 1104 include a same 2D image 1102.

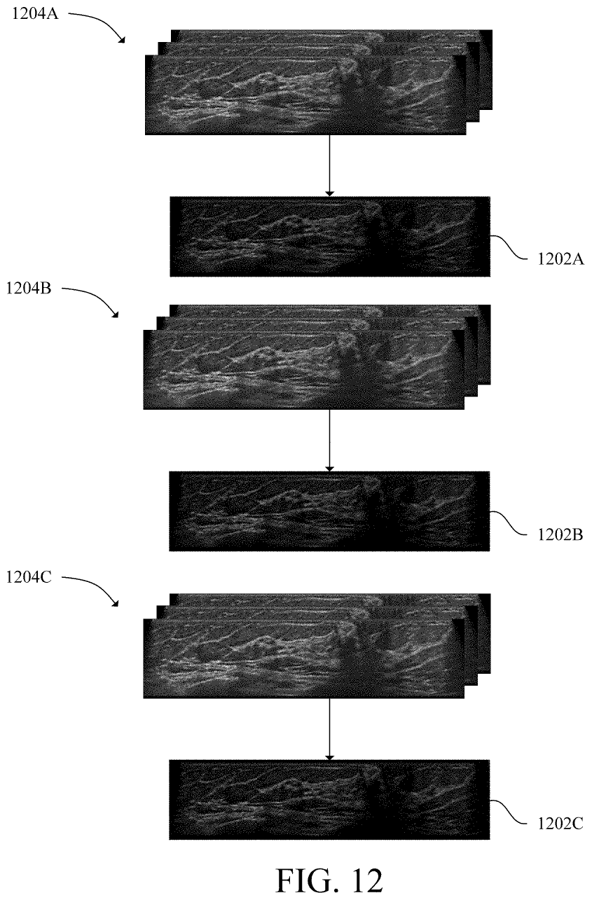

Returning to FIG. 7, at 708, the configured processor generates a projection image (i.e., minimum intensity projection image, maximum intensity projection image, average intensity projection image, median intensity projection image, etc.) from each set of 2D images. As illustrated in FIG. 12, in one embodiment, the configured processor generates a first projection image 1202A from a first set 1204A of 2D images, a second projection image 1202B from a second set 1204B of 2D images, and a third projection image 1202C from a third set 1204C of 2D images.

Returning to FIG. 7, at 710, the configured processor determines one or more regions of interest in each projection image generated at 708. In some embodiments, the configured processor may identify the regions of interest with the trained deep learning architecture. Furthermore, at 710 for each projection image generated at 708, the configured processor generates a corresponding probability map. A probability map is a derived from a projection image generated at 708 and may include one or more regions of interest. A location of a region of interest in a probability map corresponds to a location of a region of interest in a projection image generated at 708.

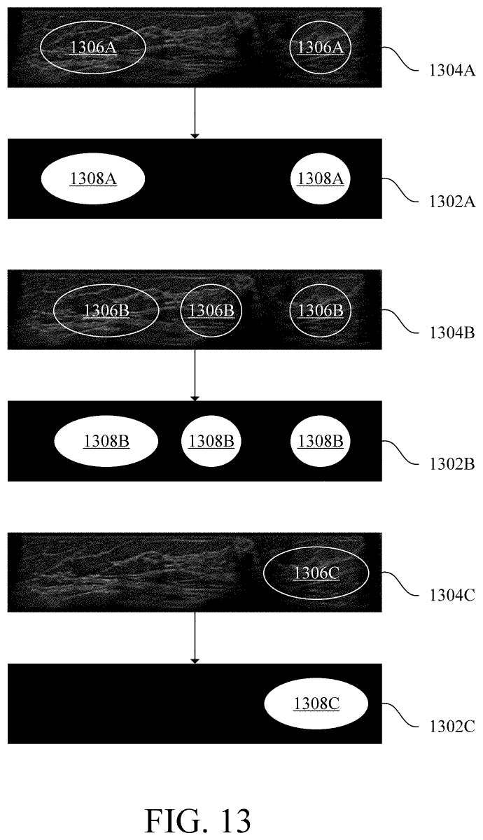

For example, FIG. 13 depicts three probability maps each generated from a different projection image. As depicted in FIG. 13 a first probability map 1302A is generated from a first projection image 1304A. In this example, configured processor identified two regions of interest 1306A in the first projection image 1304A. Accordingly, the first probability map 1302A has two regions of interest 1308A with locations that correspond to the locations of the regions of interest 1306A. As further depicted in FIG. 13, a second probability map 1302B is generated from a second projection image 1304B. In this example, the configured processor identified three regions of interest 1306B in the second projection image 1304B. Accordingly, the second probability map 1302B has three regions of interest 1308B with locations that correspond to the locations of the regions of interest 1306B. As further depicted in FIG. 13, a third probability map 1302C is generated from a third projection image 1304C. In this example, the deep learning architecture identified one region of interest 1306C in the third projection image 1304C. Accordingly, the third probability map 1302C has one region of interest 1308C with a location that correspond to the location of the regions of interest 1306C.

Furthermore, at 710, the configured processor interpolates the probability maps thereby generating a probability volume. The probability maps may correspond to a discrete slice location and as such, there may be a spatial gap between the probability maps. The configured processor interpolates space between adjacent probability maps to generate the probability volume. The configured processor may interpolate the probability maps according to a number of techniques (i.e., linear interpolation, cubic interpolation, quadratic interpolation, etc.). The probability volume includes the regions of interest that are in the probability maps.

At 712, the configured processor applies the trained deep learning architecture to the probability volume to verify that a region of interest in the probability volume is a tumor or lesion. The deep learning architecture verifies a region of interest is a tumor or lesion when the deep learning architecture determines the likelihood of the region of interest in the probability volume exceeds a threshold (i.e., 80% likely the region of interest is a tumor or lesion, 90% likely the region of interest is a tumor or lesion, 95% likely the region of interest is a tumor or lesion, etc.).

At 714, in response to verifying a region of interest is a tumor or lesion, the configured processor tags the region of interest in the probability volume. In one embodiment the configured processor tags the region of interest by highlighting the region of interest. Furthermore, at 714, the configured processor outputs a representation of the probability volume to the display 108 or the display 208.

Thus, while the information has been described above with particularity and detail in connection with what is presently deemed to be the most practical and preferred aspects, it will be apparent to those of ordinary skill in the art that numerous modifications, including, but not limited to, form, function, manner of operation, and use may be made without departing from the principles and concepts set forth herein. Also, as used herein, the examples and embodiments are meant to be illustrative only and should not be construed to be limiting in any manner.

What is claimed is:

1. A method comprising:
identifying, with a processor, a first region of interest in a first projection image;
generating, with the processor, a first probability map from the first projection image and a second probability map from a second projection image, wherein the first probability map includes a second region of interest that has location that corresponds to a location of the first region of interest;
interpolating the first probability map and the second probability map thereby generating a probability volume, wherein the probability volume includes the second region of interest; and
outputting, with the processor, a representation of the probability volume to a display.

2. The method of claim 1, further comprising:
generating the first projection image from a first set of two-dimensional images; and
generating the second projection image from a second set of two-dimensional images, wherein an automated breast ultrasound system generates the first and second set of two-dimensional images.

3. The method of claim 2, wherein the first set of two-dimensional images includes a first two-dimensional image and a second two-dimensional image and the second set of two-dimensional images includes the second two-dimensional image and a third two-dimensional image.

4. The method of claim 1, wherein the first projection image and the second projection image are minimum intensity projection images.

5. The method of claim 1, wherein the first projection image and the second projection image are maximum intensity projection images.

6. The method of claim 1, further comprising:
verifying, with a deep learning architecture that the second region of interest in the probability volume is a tumor or lesion.

7. The method of claim 6, further comprising:
in response to verifying the second region of interest in the probability volume is a tumor or lesion, tagging the second region of interest in the probability volume.

8. The method of claim 7, further comprising:
training, with the processor, the deep learning architecture with a plurality of projection training images, wherein at least one of the plurality of projection training images includes a tumor or lesion identified by a clinician.

9. A system comprising:
a medical imaging system;
a processor; and
a computer readable storage medium in communication with the processor, wherein the processor executes program instructions stored in the computer readable storage medium which cause the processor to:
receive image data from the imaging system;
generate a first and second set of two-dimensional images from the image data;
generate a first projection image from the first set of two-dimensional images and a second projection image from the second set of two-dimensional images;
identify a first region of interest in the first projection image;
generate a first probability map from the first projection image and a second probability map from a second projection image, wherein the first probability map includes a second region of interest that has location that corresponds to a location of the first region of interest;
interpolate the first probability map and the second probability map, thereby generating a probability volume, wherein the probability volume includes the second region of interest; and
output a representation of the probability volume to a display.

10. The system of claim 9, wherein the first projection image and the second projection image are minimum intensity projection images.

11. The system of claim 9, wherein the first set of two-dimensional images includes a first two-dimensional image and a second two-dimensional image and the second set of two-dimensional images includes the second two-dimensional image and a third two-dimensional image.

12. The system of claim 9, wherein the medical imaging device is an automated breast ultrasound system and the image data is ultrasound data.

13. The system of claim 12, wherein the program instructions further cause the processor to:
generate a three-dimensional volume from the ultrasound data, wherein the three-dimensional volume includes the first and second set of two-dimensional images.

14. The system of claim 9, wherein the program instructions further cause the processor to:
verify, with a deep learning architecture, that the second region of interest in the probability volume is a tumor or lesion

15. The system of claim 14, wherein the program instructions further cause the processor to:
tag the second region of interest in the probability volume in response to verifying the second region of interest is a tumor or lesion.

16. A computer readable storage medium with computer readable program instructions that, when executed by a processor, cause the processor to:
generate a three-dimensional volume from ultrasound data, wherein the three-dimensional volume includes a plurality of two-dimensional images;
separate the plurality of two-dimensional images into a first set and a second set of two-dimensional images;
generate a first projection image from the first set of two-dimensional images and a second projection image from the second set of two-dimensional images;
identify a first region of interest in the first projection image;
generate a first probability map from the first projection image and a second probability map from the second projection image, wherein the first probability map includes a second region of interest with a location that corresponds to a location of the first region of interest;
generate a probability volume from the first and second probability maps; and
identify a region of interest in the probability volume as a tumor or lesion.

17. The computer readable storage medium of claim 16, wherein the first and second projection images are minimum intensity projection images.

18. The computer readable storage medium of claim 16, wherein the first and second projection images are average intensity projection images

19. The computer readable storage medium of claim 16, wherein the first set of two-dimensional images includes a first two-dimensional image and a second two-dimensional image and the second set of two-dimensional images includes the second two-dimensional image and a third two-dimensional image.

20. The computer readable storage medium of claim 16, wherein the first and second set of two-dimensional images include a plurality of same two-dimensional images.
